(19) United States
(12) Patent Application Publication
(10) Pub. No.: US 2022/0226280 A1
(43) Pub. Date: Jul. 21, 2022

(54) COMPOSITIONS AND METHODS FOR INCREASED GLUCOSE UPTAKE AND FAT METABOLISM

(71) Applicant: Board of Supervisors of Louisiana State University and Agricultural and Mechanical College, Baton Rouge, LA (US)

(72) Inventors: Frank Greenway, Baton Rouge, LA (US); Ann Coulter, Baton Rouge, LA (US); Candida Rebello, Baton Rouge, LA (US)

(21) Appl. No.: 17/533,715

(22) Filed: Nov. 23, 2021

Related U.S. Application Data
(63) Continuation of application No. 15/989,591, filed on May 25, 2018, now abandoned.
(60) Provisional application No. 62/549,494, filed on Aug. 24, 2017.
(60) Provisional application No. 62/514,658, filed on Jun. 2, 2017.

(51) Int. Cl. A61K 31/352; A61K 31/045; A61K 8/49; A61K 8/34; A61K 36/752; A61Q 19/02; A61K 36/28; A61K 9/00; A61K 31/215; A61K 8/37; A61Q 19/08; A61K 36/534; A61K 8/60; A61Q 19/06

(52) U.S. Cl. CPC A61K 31/352; A61K 31/045; A61K 8/498; A61K 8/34; A61K 36/752; A61Q 19/02; A61Q 19/06; A61K 9/0014; A61K 31/215; A61K 8/37; A61Q 19/08; A61K 36/534; A61K 8/602; A61K 36/28

(57) ABSTRACT

Disclosed herein are compositions and methods to increase glucose uptake and fat metabolism. The disclosed composition can comprise a PI3K agonist; a TRPM8 agonist; and a pharmaceutically, nutraceutically, or cosmetically acceptable carrier. Also disclosed is a method of treating diabetes, obesity, or metabolic syndrome in a subject, comprising administering to a subject in need thereof a therapeutically effective amount of a composition comprising a PI3K agonist and a TRPM8 agonist. Also disclosed is a method of treating a skin condition in a subject, comprising topically administering to a subject in need thereof an effective amount of a composition comprising a PI3K agonist and a TRPM8 agonist.

### CROSS-REFERENCE TO RELATED APPLICATIONS

This application claims benefit of U.S. Provisional Application No. 62/514,658, filed Jun. 2, 2017, and Application Ser. No. 62/549,494, filed Aug. 24, 2017, which are hereby incorporated herein by reference in their entirety.

### BACKGROUND

Obesity is arguably the greatest public health threat in modern Western society, and it is an increasing threat throughout the world. Obesity is associated with an estimated 300,000 deaths per year. Further, numerous diseases have been correlated to obesity: Heart disease, certain types of cancer, sleep apnea, asthma, arthritis, pregnancy complications, depression and type II diabetes mellitus are all associated with excess weight. In light of the health dangers attributed to obesity, many treatments, both pharmacological and non-pharmacological, have been developed to combat this enormous problem. Non-pharmacological approaches include diet, exercise and surgical intervention. While a well-balanced diet consumed in moderation coupled with regular physical activity is the most easily applied method of controlling or losing weight, the aforementioned facts indicate that this method has not reversed the trend towards increasing obesity. Surgical intervention has been employed in conditions where obesity manifests as a real and immediate danger to a person's health. These techniques are invasive with significant inherent risks and complications. Pharmacological methods to control weight have targeted a spectrum of physiological processes. Central nervous system (CNS) appetite suppressants can cause addiction and numerous side effects including nervousness, insomnia, drowsiness, depression, nausea and lassitude. Amylase, glycosidase and lipase inhibitors have been used to prevent the absorption of fats and carbohydrates in the digestive tract, but it is virtually impossible to maintain physiological levels of these inhibitors that can effectively inhibit gastrointestinal enzymes, and therefore absorption. Therefore, new products are needed that can increase glucose uptake and fat metabolism.

### SUMMARY

Disclosed herein are compositions and methods to increase glucose uptake and fat metabolism. Naringenin is a citrus flavonoid that acts on the liver to reduce whole body cholesterol, triglycerides and insulin resistance. Dietary Naringenin activates fat oxidation and decreases fat synthesis by targeting hepatic transcriptional regulators such as PPARγ, PPARα, Pgc-1α and SREBP1/2 (Mulvihill, E. E., et al., Diabetes, 2009 58(10):2198-210; Assini, J. M., et al., Endocrinology, 2015 156(6):2087-102; Assini, J. M., et al., J Lipid Res, 2013 54(3):711-24; Goldwasser, J., et al., PLoS One, 2010 5(8):e12399; Ke, J. Y., et al., Nutr Metab (Lond), 2015 12:1; Cho, K. W., et al., Eur J Nutr, 2011 50(2):81-8; Sharma, A. K., et al., Br J Nutr, 2011 106(11):1713-23; Borradaile, N. M., et al. Diabetes, 2003 52(10):2554-61). The beneficial effects of Naringenin on blood glucose and insulin sensitivity have been shown in obese human subjects administered grapefruit juice three times a day. After 12 weeks, there was a significant reduction in insulin that was associated with weight loss (Fujioka, K., et al., J Med Food, 2006 9(1):49-54). Research examining the mechanism underlying reduction in blood glucose by Naringenin has demonstrated a role for activation of PI3K, IRS1, PPARγ and inhibition of PEPCK in liver and liver cell lines (Sharma, A. K., et al., Br J Nutr, 2011 106(11):1713-23; Borradaile, N. M., et al. Diabetes, 2003 52(10):2554-61; Kannappan, S. and C. V. Anuradha, Eur J Nutr, 2010 49(2):101-9; Park, H. J., et al., J Nutr Biochem, 2013 24(2):419-27). Enhanced glucose uptake with activation of AMPK after Naringenin treatment was observed in muscle cells (Zygmunt, K., et al., Biochem Biophys Res Commun, 2010 398(2):178-83).

As disclosed herein, transient receptor potential cation channel subfamily M member 8 (TRPM8) agonists, working through pip2, can prevent the down regulation of the body's response to phosphatidylinositol-4,5-bisphosphate 3-kinase (PI3K) agonists, such as naringenin. Therefore, disclosed herein is a composition comprising a PI3K agonist; a TRPM8 agonist; and a pharmaceutically, nutraceutically, or cosmetically acceptable carrier.

The TRPM8 agonist can be natural or synthetic. For example, the TRPM8 agonist can be menthol. In some embodiments, the TRPM8 agonist comprises eucalyptol, linalool and givaudan cooling flavor (QB-113-979-5, Dubendorf, Switzeraland) (Bautista D. M. et al. Nature 2007 448:204-208; Michlig S et al. Sci Rep. 2016 6:20759). In some embodiments, the TRPM8 agonist comprises 3-lodothyronamine (Lucius A et al. Cell Signal. 2016 28(3):136-47). In some embodiments, the TRPM8 agonist comprises camphor (Selescu T et al. Chem Senses. 2013 38(7):563-75). In some embodiments, the TRPM8 agonist comprises 1,8-cineole, 1,4-cineole, or a combination thereof (Takaishi M, et al. Mol Pain. 2012 8:86). Additional TRPM8 agonists include synthetic p-menthane carboxamides along with other class of compounds such as aliphatic/alicyclic alcohols/esters/amides, sulphones/sulphoxides/sulphonamides, heterocyclics, keto-enamines/lactams, and phosphine oxides (Bharate S S and Bharate S B. ACS Chem Neurosci. 2012 3(4):248-67; Ma S et al. Pak J Pharm Sci. 2008 21(4):370-8). Icilin is a synthetic agonist of TRPM8; however, in some embodiments, the TRPM8 agonist comprises a menthol that is derived from natural sources, such as a mint plant or sunflower essential oil.

The PI3K agonist can also be natural or synthetic. In some embodiments, the PI3K agonist comprises naringenin or naringin. In some embodiments, the PI3K agonist is selected from the group consisting of butin, Eriodictyol, hesperetin, hesperidin, homoeriodictyol, and isosakuranetin.

In preferred embodiments, the TRPM8 agonist comprises menthol and the PI3K agonist comprises naringenin. As shown herein, naringenin and menthol synergize to induce UCP1 mRNA expression.

In some embodiments, the menthol is in an amount of between about 1 μM to about 50 μM, about 1 μM to about 10 μM, about 10 μM to about 30 μM, about 30 μM to about 50 μM, including about 1, 2, 3, 4, 5, 6, 7, 8, 9, 10, 11, 12, 13, 14, 15, 16, 17, 18, 19, 20, 25, 30, 35, 40, 45, or 50 μM.

In some embodiments, the naringenin is in an amount of between about 0.1 μM to about 50 μM, about 1 μM to about 10 μM, about 5 μM to about 30 μM, about 30 μM to about 50 μM, including about 1, 2, 3, 4, 5, 6, 7, 8, 9, 10, 11, 12, 13, 14, 15, 16, 17, 18, 19, 20, 25, 30, 35, 40, 45, or 50 μM. In some embodiments, the weight ratio of menthol agonist to naringenin in the disclosed composition is about 10:1 to about 1:10, including about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1.10.

Also disclosed is a method of treating diabetes, obesity, or metabolic syndrome in a subject, comprising administering to a subject in need thereof a therapeutically effective amount of a composition comprising a PI3K agonist and a TRPM8 agonist (e.g. naringenin and menthol). In some embodiments, the disclosed composition is selectively administered to body locations of the subject where fat loss is preferred. In some embodiments, the subject can be undergoing a weight loss program, such as diet, exercise, behavior modification, surgery, devices that reduce obesity (e.g. lap band, gastric balloons, V-Bloc transpyloric shuttle, and Aspire™), obesity medications, obesity supplements, or a combination thereof.

Also disclosed is a method of treating a skin condition in a subject, comprising topically administering to a subject in need thereof an effective amount of a composition comprising a PI3K agonist and a TRPM8 agonist (e.g. naringenin and menthol). In some embodiments, the composition further comprises retinol or a retinyl ester. For example, the retinyl ester can be selected from the group consisting of retinyl palmitate, retinyl acetate, retinyl propionate, retinyl linoleate, and mixtures thereof. The disclosed method can be used to treat a variety of skin conditions, including, but not limited to, dry skin, photodamaged skin, appearance of wrinkles, age spots, aged skin, acne, skin lightening, psoriasis, atopic dermatosis, and sebum secretion. In some embodiments, the disclosed method can be used to treat sunburn, itching, inflammation, skin aging, atopic dermatitis aches, skin cancer, pains, razor burn, congestion halitosis, or sore throat. In some embodiments, the disclosed method can be used as an antispasmodic/smooth muscle relaxant in gastrointestinal endoscopy.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### DESCRIPTION OF DRAWINGS

Further aspects of the present disclosure will be more readily appreciated upon review of the detailed description of its various embodiments, described below, when taken in conjunction with the accompanying drawings.

FIG. 1 shows mRNA induction of differentiated human adipocytes treated with 10 μM Naringenin for 7 days. Gene expression was analyzed using Taqman RT-PCR and values are expressed as fold increase over untreated controls (n=5).

FIG. 2A shows UCP1 mRNA induction of differentiated human adipocytes treated for 6 days. Gene expression was analyzed using Taqman RT-PCR and values are expressed as fold increase over untreated controls. Naringenin and Menthol v. Control: p=0.03, Naringenin and Menthol v. Naringenin: p=0.001. Naringenin and Menthol v. Menthol: p=0.06, Naringenin v. control: p>0.05, menthol v. control: P>0.05.

FIG. 2B shows Glut 4 mRNA induction of differentiated human adipocytes treated for 6 days. Gene expression was analyzed using Taqman RT-PCR and values are expressed as fold increase over untreated controls. Naringenin and Menthol v. Control: p=0.008, Naringenin and Menthol v. Naringenin: p=0.01. Naringenin and Menthol v. Menthol: p<0.001, Naringenin v. Control: p>0.05, Menthol v. Control: p>0.05.

FIG. 3 provides results of human adipocytes treated with 10 μM Naringenin for 3 days in the presence of inhibitors and expression of UCP1 mRNA was analyzed using Taqman RT-PCR.

FIG. 4 is a bar graph showing fold increase of cells treated for 7 d with Naringenin (Nar) alone or Naringenin and Menthol (Nar+Men). Concentrations are in μM. Synergy observed as low as 1 μM Naringenin and 5 μM Menthol.

FIG. 5 is a bar graph showing effect of TRPM3-blocking flavanones on cell viability. From Straub I, et al. Mol Pharmacol 2013; 84:736-750. HEK293 cells were incubated with different concentrations of TRPM3-blocking flavanones, and cell viability was detected by performing a MTT viability test. Data represent the mean values±S.E.M. of 5-8 independent experiments. To test for statistically significant differences, analysis of variance with Tukey's post-hoc test was performed with the normalized data (*P<0.05; **P<0.01).

FIG. 6 is a bar graph showing fold increase of UCP1 mRNA in cells treated for 7 d with 5 μM Naringenin (Nar) alone, 5 μM Naringenin and 0.1 μM Menthol, or 5 μM Naringenin and 1 μM Menthol.

FIG. 7 is a bar graph showing fold increase of UCP1 mRNA in cells treated for 7 d with 5 μM Naringenin (Nar) alone, 0.1 μM Naringenin and 30 μM Menthol, 1 μM Naringenin and 1 μM Menthol, or 1 μM Naringenin and 5 μM Menthol.

FIG. 8 is a bar graph showing fold increase of UCP1 mRNA in cells treated for 7 d with 5 μM Naringenin (Nar) alone, 5 μM Naringenin and 100 μM eucaluptol, or 5 μM Naringenin and 200 μM eucaluptol.

FIG. 9 is a bar graph showing fold increase of UCP1 mRNA in cells treated for 7 d with 5 μM Naringenin (Nar) alone, 0.5 μM icilin, or 5 μM Naringenin and 0.5 μM icilin.

FIG. 10 is a bar graph showing fold increase of UCP1 mRNA in cells treated for 7 d with 5 μM Hesperetin, Apigenin, Quercetin, or Cyaniding, each with and without 20 μM Menthol.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Compositions

Disclosed herein is a composition comprising a PI3K agonist; a TRPM8 agonist; and a pharmaceutically, nutraceutically, or cosmetically acceptable carrier.

The TRPM8 agonist can be natural or synthetic. For example, the TRPM8 agonist can be menthol or icilin. Icilin is a synthetic agonist of TRPM8; however, menthol can also be derived from natural sources, such as a mint plant or sunflower essential oil.

The PI3K agonist can also be natural or synthetic. In some embodiments, the PI3K agonist comprises naringenin or naringin.

Naringenin (2,3-dihydro-5,7-dihydroxy-2-(4-hydroxyphenyl)-4H-1-benzopyran-4-one) is the pharmacologically functioning structural moiety of naringin. The structures of naringenin and naringin are as follows:

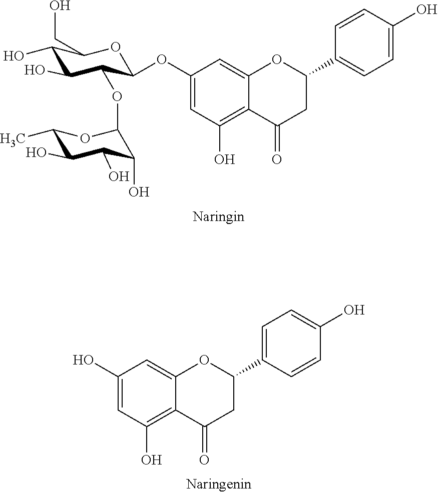

The naringenin can be a single stereoisomer (2S-naringenin or 2R-naringenin), a stereoisomer mixture, a salt of a single stereoisomer, a salt of a stereoisomer mixture or a mixture thereof. Further, the naringenin can be an isolated and purified form or a botanical extract. For example, isolated and purified naringenin can be obtained by synthetic methods; by de novo production, e.g., in Saccharomyces cerevisiae (Koopman, et al. (2012) Microbial Cell Factorizes 11:155); or from commercial sources (e.g., Sigma-Aldrich).

If provided as a botanical extract, preferably the extract is enriched for naringenin to achieve a content of about 15% and greater, for example, from about 15% to about 95%, from about 60% to about 95% or from about 70% to about 95%. Unless otherwise specified, percentages (% s) are by weight. Naringenin botanical extracts can be obtained by conventional methods from various plant sources including, for example, but not limited to, tomato (Yoshimura, et al. (2007) Allergol. Int. 56:225-230), citrus such as Citrus aurantium, C. grandis, C. junos or C. paradisi (Heo, et al. (2004) Dement. Geriatr. Cogn. Discord. 17:151-157; Coffin (1971) J. Agr. Food Chem. 19:513) or Mentha aquatica L. (Olsen, et al. (2008) J. Ethnopharmacol. 117:500-2); or from commercial sources such as Xiamen JieJing Biology Technology Co., Ltd (Xiamen, China), which provides a naringenin extract from Citrus paradisi Macfadyen. Extracts containing naringin or naringenin-7-glucoside (glycosidic forms of naringenin) can be treated via chemical or biological methods to release the aglycone using, e.g., acid hydrolysis (Pulley & von Loesecke (1939) J. Am. Chem. Soc. 61:175) or enzymatic hydrolysis (Ferreira, et al. (2008) Food Technol. Biotechnol. 46:146-150; Thomas, et al. (2006) J. Food Sci. 23:591-98).

Naringenin is soluble in ethanol, ether, oils (e.g., olive oil or citrus oil) and supercritical carbon dioxide and is sparingly soluble in aqueous solutions. Accordingly, in some embodiments, the naringenin is provided as a water-soluble salt. Salts include base addition salts which may be formed when acidic protons present are capable of reacting with inorganic or organic bases. Acceptable inorganic bases include, for example, but not limited to, sodium hydroxide, sodium carbonate, potassium hydroxide, aluminum hydroxide and calcium hydroxide. Acceptable organic bases include, for example, but not limited to, ethanolamine, diethanolamine, triethanolamine, tromethamine, N-methylglucamine and the like. Salts of naringenin can be prepared by conventional methods or can be obtained commercially. For example, a water-soluble potassium salt of naringenin is provided by Natrafu S A (Alcantarilla, Spain). The naringenin can be naringenin, a naringenin salt, or a combination thereof. Water-soluble naringenin-amino acid esters (Kim, et al. (2005) Bull. Korenn Chem. Soc. 26:2065) or naringenin in complex with hydroxypropyl-β-cyclodextrin (Wen, et al. (2010) Molecules 15:4401-7; 2011/0312985) can also be used.

In some embodiments, the TRPM8 agonist is in an amount of between about 1 μM to about 50 μM, about 1 μM to about 10 μM, about 10 μM to about 30 μM, about 30 μM to about 50 μM, including about 1, 2, 3, 4, 5, 6, 7, 8, 9, 10, 11, 12, 13, 14, 15, 16, 17, 18, 19, 20, 25, 30, 35, 40, 45, or 50 μM. In some embodiments, the PI3K agonist is in an amount of between about 1 μM to about 50 μM, about 1 μM to about 10 μM, about 10 μM to about 30 μM, about 30 μM to about 50 μM, including about 1, 2, 3, 4, 5, 6, 7, 8, 9, 10, 11, 12, 13, 14, 15, 16, 17, 18, 19, 20, 25, 30, 35, 40, 45, or 50 μM. In some embodiments, the weight ratio of TRPM8 agonist to PI3K agonist in the disclosed composition is about 10:1 to about 1:10, including about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10.

The compositions disclosed herein can be used therapeutically in combination with a pharmaceutically, nutraceutically, or cosmetically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Compositions may also include carriers, thickeners, diluents, buffers, preservatives, surface active agents and the like in addition to the molecule of choice.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration. Topical administration is contemplated for transdermal delivery and also for administration to the eyes or mucosa, or for inhalation therapies. Nasal solutions of the active compound alone or in combination with other pharmaceutically acceptable excipients can also be administered.

Compositions for oral administration include powders or granules, suspensions or solutions in water or non-aqueous media, capsules, sachets, or tablets. Thickeners, flavorings, diluents, emulsifiers, dispersing aids or binders may be desirable. Oral pharmaceutical dosage forms are either solid, gel or liquid. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Methods

Disclosed herein is a method of treating diabetes, obesity, or metabolic syndrome in a subject, comprising administering to a subject in need thereof a therapeutically effective amount of a composition comprising a PI3K agonist and a TRPM8 agonist. In some embodiments, the disclosed composition is selectively administered to body locations of the subject where fat loss is preferred. In some embodiments, the subject can be undergoing a weight loss program, such as diet, exercise, surgery, obesity medications, or a combination thereof.

Also disclosed is a method of treating a skin condition in a subject, comprising topically administering to a subject in need thereof an effective amount of a composition comprising a PI3K agonist and a TRPM8 agonist. In some embodiments, the composition further comprises retinol or a retinyl ester. For example, the retinyl ester can be selected from the group consisting of retinyl palmitate, retinyl acetate, retinyl propionate, retinyl linoleate, and mixtures thereof. The disclosed method can be used to treat a variety of skin conditions, including, but not limited to, dry skin, photodamaged skin, appearance of wrinkles, age spots, aged skin, acne, skin lightening, psoriasis, atopic dermatosis, and sebum secretion.

The herein disclosed compositions, including pharmaceutical composition, may be administered in a number of ways depending on whether local or systemic treatment is desired, and on the area to be treated. For example, the disclosed compositions can be administered orally, parenterally, transdermally, ophthalmically, vaginally, rectally, intranasally, or by inhalation.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

Naringenin is a citrus flavonoid that acts on the liver to reduce whole body cholesterol, triglycerides and insulin resistance. A growing number of studies in obese rodent models have shown that dietary Naringenin activates fat oxidation and decreases fat synthesis by targeting hepatic transcriptional regulators such as PPARγ, PPARα, Pgc-1α and SREBP1/2 (Mulvihill, E. E., et al., Diabetes, 2009 58(10):2198-210; Assini, J. M., et al., Endocrinology, 2015 156(6):2087-102; Assini, J. M., et al., J Lipid Res, 2013 54(3):711-24; Goldwasser, J., et al., PLoS One, 2010 5(8):e12399; Ke, J. Y., et al., Nutr Metab (Lond), 2015 12:1; Cho, K. W., et al., Eur J Nutr, 2011 50(2):81-8; Sharma, A. K., et al., Br J Nutr, 2011 106(11):1713-23; Borradaile, N. M., et al. Diabetes, 2003 52(10):2554-61). The beneficial effects of Naringenin on blood insulin and insulin sensitivity have been shown in obese human subjects administered grapefruit juice three times a day. After 12 weeks, there was a significant reduction in insulin that was associated with weight loss (Fujioka, K., et al., J Med Food, 2006 9(1):49-54). Research examining the mechanism underlying reduction in blood glucose by Naringenin has demonstrated a role for activation of PI3K, IRS1, PPARγ and inhibition of PEPCK in liver and liver cell lines (Sharma, A. K., et al., Br J Nutr, 2011 106(11):1713-23; Borradaile, N. M., et al. Diabetes, 2003 52(10):2554-61; Kannappan, S. and C. V. Anuradha, Eur J Nutr, 2010 49(2):101-9; Park, H. J., et al., J Nutr Biochem, 2013 24(2):419-27). Enhanced glucose uptake with activation of AMPK after Naringenin treatment was observed in muscle cells (Zygmunt, K., et al., Biochem Biophys Res Commun, 2010 398(2):178-83).

Naringenin has also been shown to inhibit 11-beta hydroxysteroid dehydrogenase type 1 (11-beta HSD-1) in fat tissue, an enzyme that converts inactive precursor cortisone into active cortisol. Activity of 11-beta HSD-1 has been associated with the metabolic syndrome. Naringenin also inhibits 11-beta hydroxysteroid dehydrogenase type 2 (11-beta HSD-2) in the kidney which converts active cortisol to inactive cortisone. Inhibition of 11-beta HSD-2 can result in a mineralocorticoid type of hypertension. The study of grapefruit in humans that gave weight loss and improved insulin sensitivity gave 234 mL of grapefruit juice three times a day (Fujioka, K., et al., J Med Food, 2006 9(1):49-54). A study that used 1 quart of grapefruit juice per day found that one could detect an inhibition of 11-beta HSD-2 by cortisone to cortisol ratios, but there was no difference in potassium clearance (Lee, Y. S., et al., Clin Pharmacol Ther, 1996 59(1):62-71). Another study gave 1 to 2 liters of grapefruit juice per day and although there was evidence of inhibition of 11-beta HSD by the cortisone to cortisol ratios, there was no increase in urine potassium (Reidenberg, M. M., Toxicology, 2000 144(1-3):107-11). A case report did document evidence of mineralocorticoid excess in a woman who drank a liter of grapefruit juice a day that went away when the grapefruit juice was stopped suggesting that in a sensitive person, 11-beta HSD-2 can be inhibited sufficiently to increase urinary potassium (Palermo, M., et al., Clin Endocrinol (Oxf), 2003 59(1):143-4).

Cortisol causes thinning of the skin, atrophy, impaired wound healing and 11-beta HSD-1 is increased in ageing human skin. Topical treatment to the skin with an inhibitor of 11-beta HSD-1 accelerated wound healing and improved age-associated impairments in dermal integrity (Tiganescu, A., et al., J Clin Invest, 2013 123(7):3051-60).

A limited number of studies have examined the direct effects of Naringenin on adipose tissue in obese rodents. Naringenin treatment reduces adipocyte hypertrophy, macrophage invasion, inflammation and production of inflammatory cytokines (Assini, J. M., et al., J Lipid Res, 2013 54(3):711-24; Ke, J. Y., et al., Nutr Metab (Lond), 2015 12:1; Yoshida, H., et al., Biochem Biophys Res Commun, 2014 454(1):95-101; Yoshida, H., et al., J Nutr Biochem, 2013 24(7):1276-84).

Flavonoids such as Naringenin are biotransformed into their metabolites by gut microbiota; however, flavonoids also modulate the composition of the gut microbial community. The formation of citrus flavonoids and the modulation of gut microbiota may both contribute to the health benefits they confer (Cassidy, A. and A. M. Minihane, Am J Clin Nutr, 2017 105(1):10-22). Examination of fecal metabolomic profiles suggest that Naringenin is depleted in mice fed a high fat diet as a result of a stimulation of microbiome mediated flavonoid degrading capacity. In these mice, the addition of Narigenin to the high fat diet attenuated weight gain by induction of the major thermogenic factor uncoupling protein 1 (UCP1) in brown adipose tissue (Thaiss, C. A., et al., Nature, 2016 540:544-551).

### Example 1. Screen for Natural Compounds to Treat Insulin Resistance and Obesity

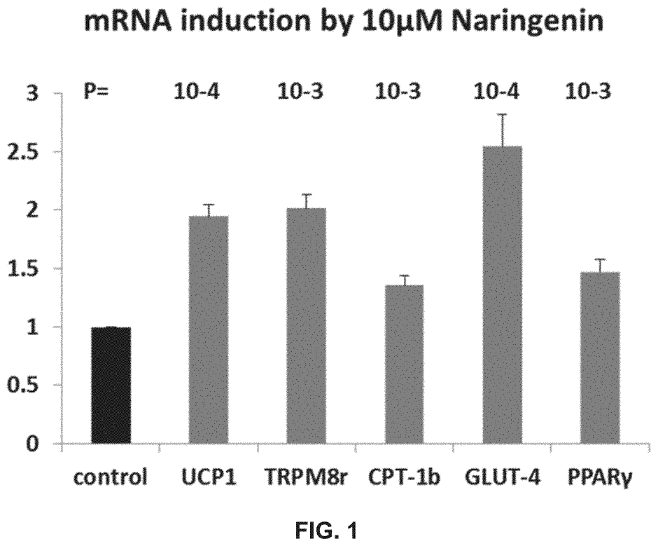

A screen was conducted for natural compounds to treat insulin resistance and obesity, and we have discovered that Naringenin significantly induces the brown adipocyte markers Uncoupling Protein 1 (UCP1), Glucose transporter type 4 (GLUT4) and carnitine palmitoyltransferase 1 (CPT-1) in human subcutaneous adipocyte cultures. UCP1 mRNA levels increased 4-8 fold in human subcutaneous adipocytes treated from 4-7 days with Naringenin at a concentration of 10 μM, the Cmax detected in human plasma after intake of citrus beverages (FIG. 1) (Kanaze, F. I., et al., Eur J Clin Nutr, 2007 61(4):472-7). No increases were observed in the transcriptional activator Pgc-1α that regulates Ucp1 levels in rodent brown adipocytes.

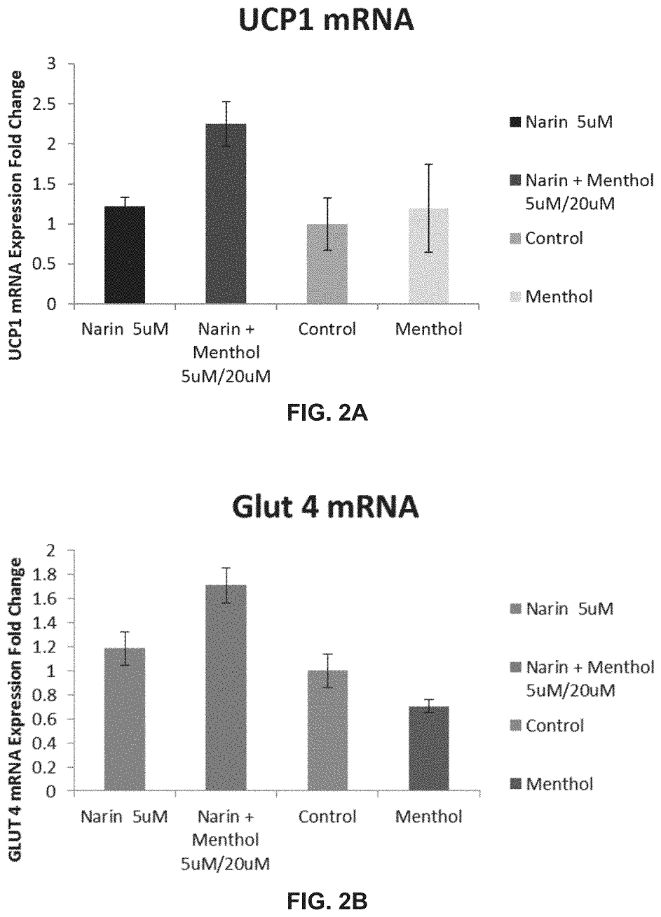

Treatment of human adipocytes under similar conditions with the combination of 20 μM Menthol (at Cmax) (Valente, A., et al., Food Chem Toxicol, 2015 86:262-73) and 5 μM Naringenin synergistically enhanced induction of UCP1 and GLUT4 mRNA levels (FIGS. 2A-B). Since Naringenin has challenges with solubility that affect its efficacy, attempts have been made to overcome this by complexation with β-cyclodextrin, altering the form in which it is consumed, increasing solubility by removing the rutinoside moiety enzymatically and optimizing absorption by gut microbiota (Kanaze, F. I., et al., Eur J Clin Nutr, 2007 61(4):472-7; Vallejo, F., et al., J Agric Food Chem, 2010 58(10):6516-24; Erlund, I., et al., J Nutr, 2001 131(2):235-41; Shulman, M., et al., PLoS One, 2011 6(4):e18033; Brett, G. M., et al., Br J Nutr, 2009 101(5):664-75). The synergy with menthol, by reducing the dose needed to obtain efficacy, is another method of increasing efficacy that is an alternative to increasing solubility.

Menthol is a specific agonist for the TRPM8 receptor, a G-protein coupled calcium channel expressed in cold-sensing peripheral neurons, adipocytes, prostate and liver (Klasen, K., et al., Pflugers Arch, 2012 463(6):779-97; Yudin, Y. and T. Rohacs, Mol Cell Endocrinol, 2012 353(1-2):68-74; Malkia, A., et al. Curr Pharm Biotechnol, 2011 12(1):54-67; Fonfria, E., et al., J Recept Signal Transduct Res, 2006 26(3):159-78) TRPM8 receptors are a key component of the body's ability to detect cold (Bautista, D. M., et al., Nature, 2007 448(7150):204-8; Colburn, R. W., et al., Neuron, 2007 54(3):379-86; Knowlton, W. M., et al., PLoS One, 2011 6(9):e25894; Dhaka, A., et al., Neuron, 2007 54(3):371-8; Ma, S., et al., J Mol Cell Biol, 2012 4(2):88-96). Adipocytes have the ability to sense cold independently of the nervous system, and ex vivo human adipocytes exposed to 28° C. activate thermogenic gene expression independently of neural adrenergic pathways (Ye, L., et al., Proc Natl Acad Sci USA, 2013 110(30):12480-5). One study showed that treatment of human adipocytes in culture with extremely high concentrations of Menthol (100 μM) overnight activated a 1.5 fold increase in UCP1 expression, and this effect was enhanced to 40-fold when cells were exposed to a cooler temperature of 26° C. (Rossato, M., et al., Mol Cell Endocrinol, 2014 383(1-2):137-46). In brown adipocytes from mice, Menthol induced a concentration-dependent increase in Ucp1 expression (Ma, S., et al., J Mol Cell Biol, 2012 4(2):88-96). This effect was absent in cells from TRPM8 receptor knockout mice.

The synergy between Naringenin and Menthol to induce UCP1 expression suggests that the TRPM8 receptor is involved and activity of the TRPM8 receptor is increased relative to that of Menthol alone. Indeed, we have observed that 10 μM Naringenin alone induces a 4-fold increase in TRPM8 receptor expression and induction of receptor levels is even higher with the combination of Naringenin plus Menthol.

Binding of Menthol to the TRPM8 receptor initiates an influx of extracellular Ca²⁺ and a cascade of signaling events mediated by Gaq (Klasen, K., et al., Pflugers Arch, 2012 463(6):779-97; Zhang, X., et al., Nat Cell Biol, 2012 14(8):851-8). Hydrolysis of Phosphatidylinositol 4,5 bisphosphate (PIP2) by Phospholipase C (PLC) releases the second messengers diacylglycerol (DG) and inositol 1, 4, 5-trisphosphate (IP3). IP3 subsequently signals release of intracellular calcium stores and DG activates Protein Kinase C. The TRPM8 receptor requires membrane PIP2 for activation, and cold and Menthol function as allosteric activators that sensitize the receptor to PIP2 (Yudin, Y. and T. Rohacs, Mol Cell Endocrinol, 2012 353(1-2):68-74; Yudin, Y., et al., J Physiol, 2011 589(Pt 24):6007-27; Rohacs, T., et al., Nat Neurosci, 2005 8(5):626-34; Rohacs, T., Handb Exp Pharmacol, 2014 223:1143-76). After stimulation with Menthol, rapid receptor desensitization occurs due to depletion of PIP2 by PLC (Abe, J., et al., Neurosci Lett, 2006 397(1-2):140-4; Daniels, R. L., J Biol Chem, 2009 284(3):1570-82). Repeated Menthol applications cause TRPM8 receptor binding and downregulation by Ca²⁺ -calmodulin (Sarria, I., et al., J Neurophysiol, 2011 106(6):3056-66). No induction of UCP1 or other beige markers was observed after treatment of human adipocyte cultures with Menthol alone (FIGS. 2A-B), possibly due to desensitization.

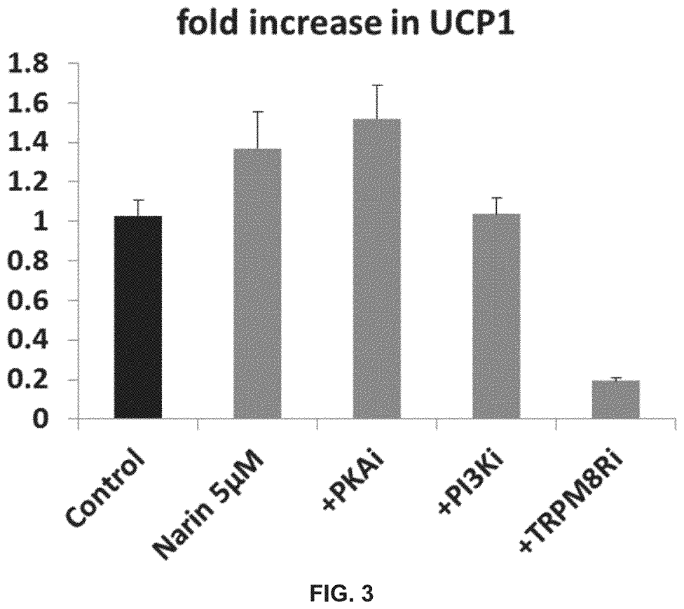

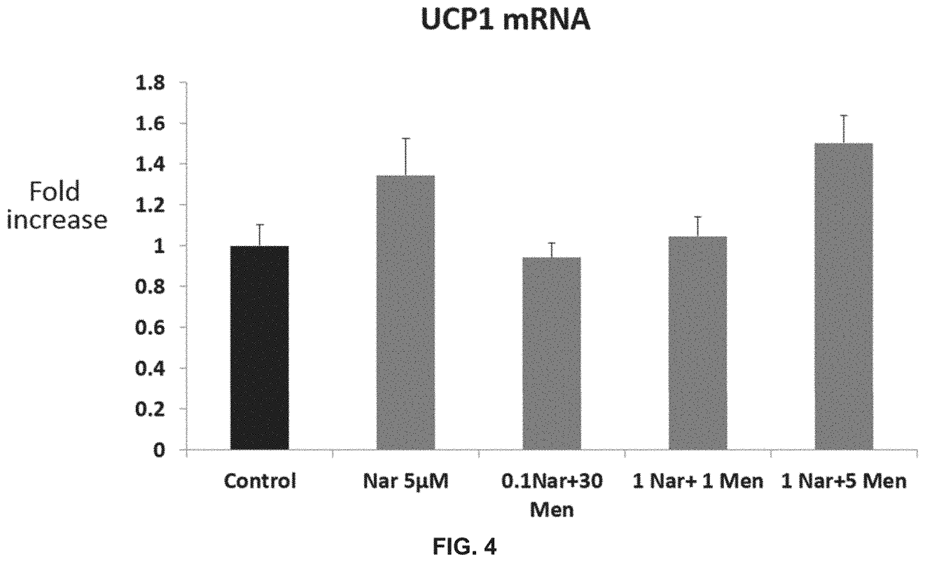

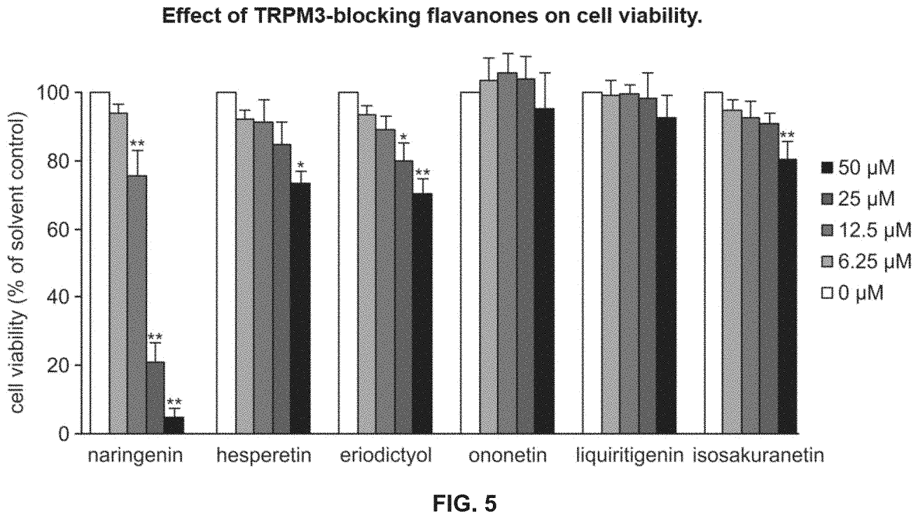

Naringenin activates the PI3 kinase/Akt (PI3K) signaling pathway in liver, brain and other cell types (Borradaile, N. M., et al. Diabetes, 2003 52(10):2554-61; Kannappan, S. and C. V. Anuradha, Eur J Nutr, 2010 49(2):101-9; Hua, F. Z., et al., Int J Mol Med, 2016 38(4):1271-80; Kulasekaran, G. and S. Ganapasam, Mol Cell Biochem, 2015 409(1-2):199-211; Wu, J. B., et al., Eur J Pharmacol, 2008 588(2-3):333-41; Lim, W. and G. Song, Mol Cell Endocrinol, 2016 428:28-37; Lim, W., et al., J Cell Biochem, 2016 118(5):1118-1131). PI3K produces PIP2 and PIP3, coactivators of the TRPM8 receptor that prevent desensitization (Yudin, Y., et al., J Physiol, 2011 589(Pt 24):6007-27; Rohacs, T., et al., Nat Neurosci, 2005 8(5):626-34). To identify the mechanism of UCP1 induction by Naringenin, cells were treated with inhibitors of PKA (H89) PI3K (LY294002) and TRPM8 receptor (PBMC) during 5 μM Naringenin treatment for 3 days. Both the PI3Ki and TRPM8Ri prevented Naringenin-stimulated UCP1 expression, suggesting that PI3K activation and TRPM8 receptor activity are both required for UCP1 induction (FIG. 3).

Naringenin supplementation significantly lowers serum cholesterol and reduces HMG-CoA reductase activity (cellular cholesterol synthesis). Membrane cholesterol levels influence the subcellular localization and activity of the TRPM8 receptor (Malkia, A., et al. Curr Pharm Biotechnol, 2011 12(1):54-67). The TRPM8 receptor is sequestered in cholesterol-rich lipid rafts and vesicles (Veliz, L. A., et al., PLoS One, 2010 5(10):e13290; Toro, C. A., et al., J Neurosci, 2015 35(2):571-82; Morenilla-Palao, C., et al., J Biot Chem, 2009 284(14):9215-24) where it is inactive. The cholesterol-lowering effect of Naringenin is consistent with stabilization and enhanced activity of TRPM8 receptors in the plasma membrane.

### Example 2. Analysis of Concentration Ranges of Naringenin and Menthol for Synergy

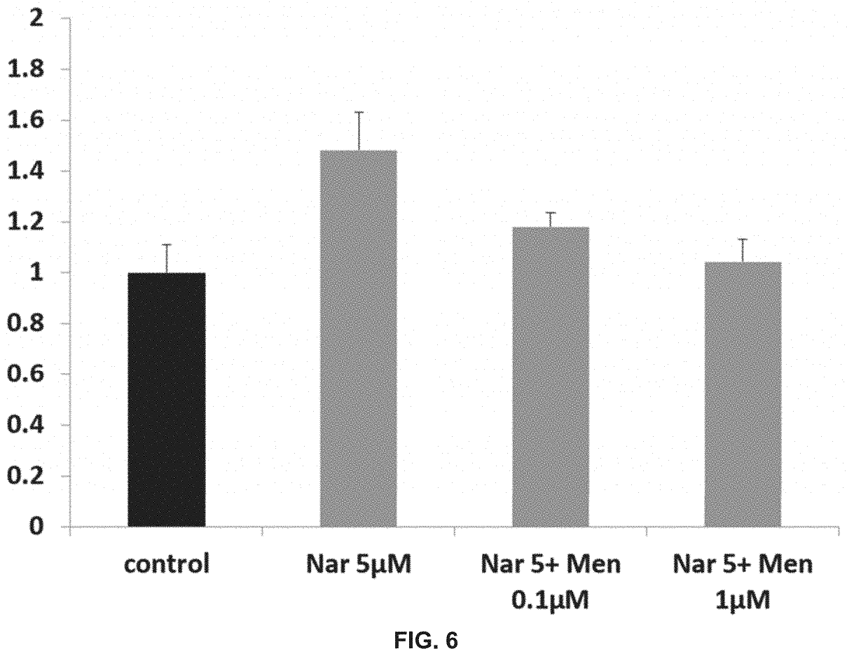

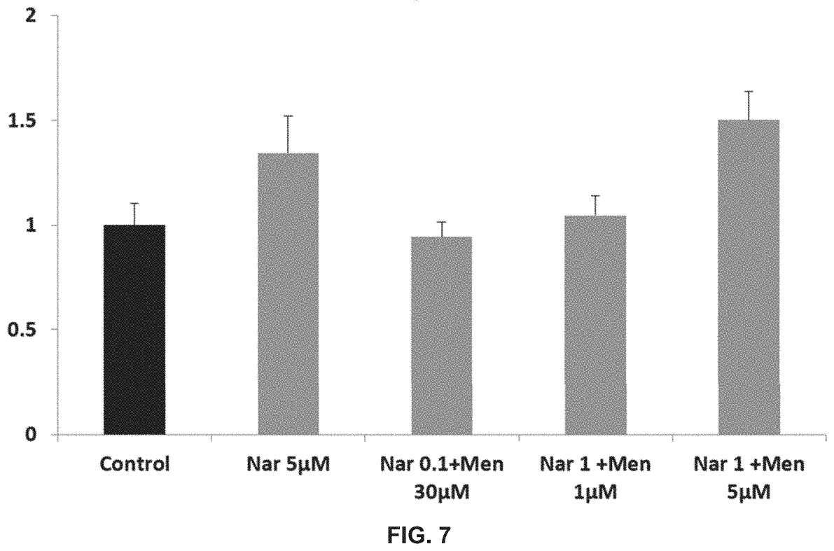

The maximum concentration for treatment of adipocyte cultures by naringenin is limited by toxicity to cells (12.5 μM) (Straub I, et al. Mol Pharmacol 2013 84:736-50) and achievable serum Cₘₐₓ of 10 μM (Kanaze F I, et al. Eur J Clin Nutr 2007 61:472-7). To determine the lowest concentrations with efficacy to induce UCP1 mRNA in human adipocytes over a 7 day time period, cells were treated with multiple concentrations of menthol combined with naringenin at 5 μM (FIG. 6). Cells were also treated with lower concentrations of naringenin combined with up to 30 μM menthol, the high end of serum Cmax for menthol (FIG. 7).

As shown in FIG. 6, 1 μM menthol does not induce UCP1 mRNA expression in combination with 5 μM naringenin. As shown in FIG. 7, 0.1 μM naringenin does not induce UCP1 mRNA expression even with the maximum level of menthol, 30 μM. Therefore, the naringenin plus menthol synergy to induce UCP1 mRNA expression occurs when naringenin is higher than 0.1 μM and menthol is higher than 1 μM.

### Example 3. Analysis of Synergy Between Naringenin and Other TRPM8 Activating Compounds in the Induction of UCP1, in Human Adipocytes

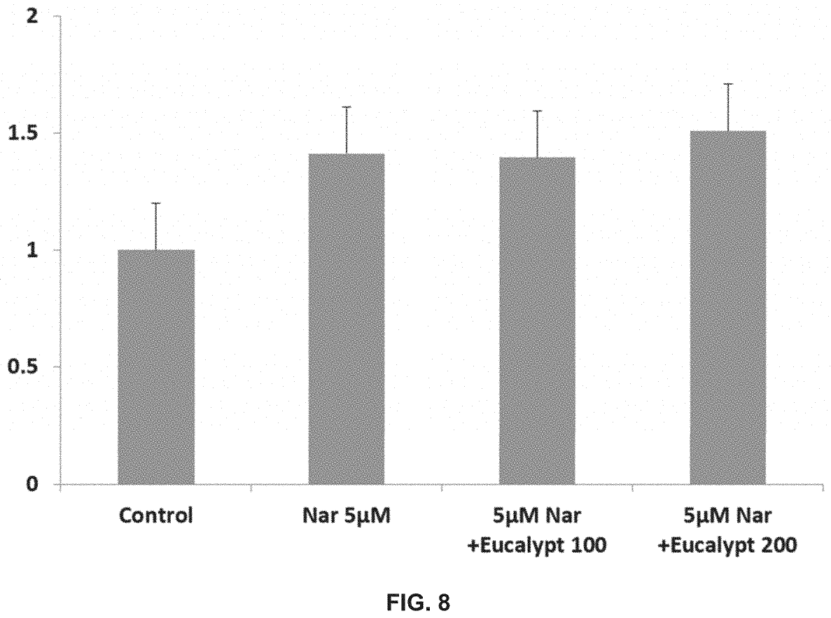

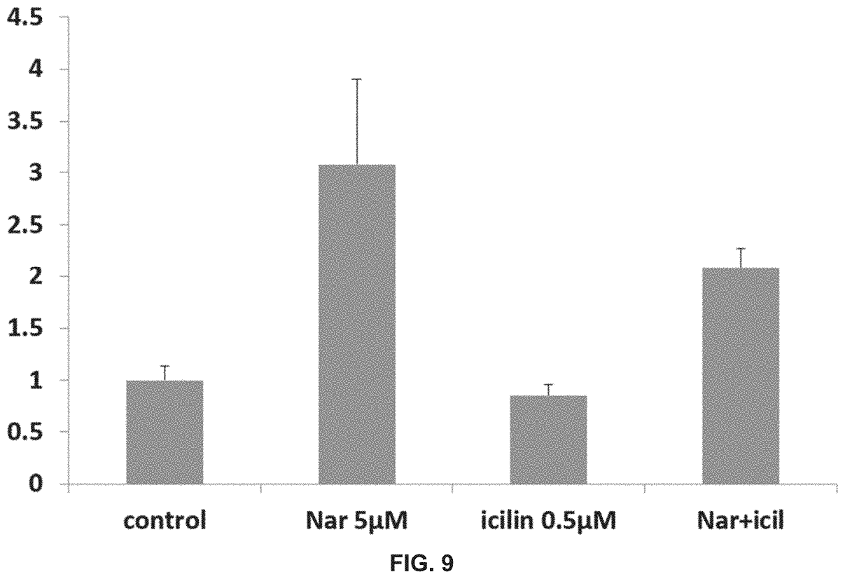

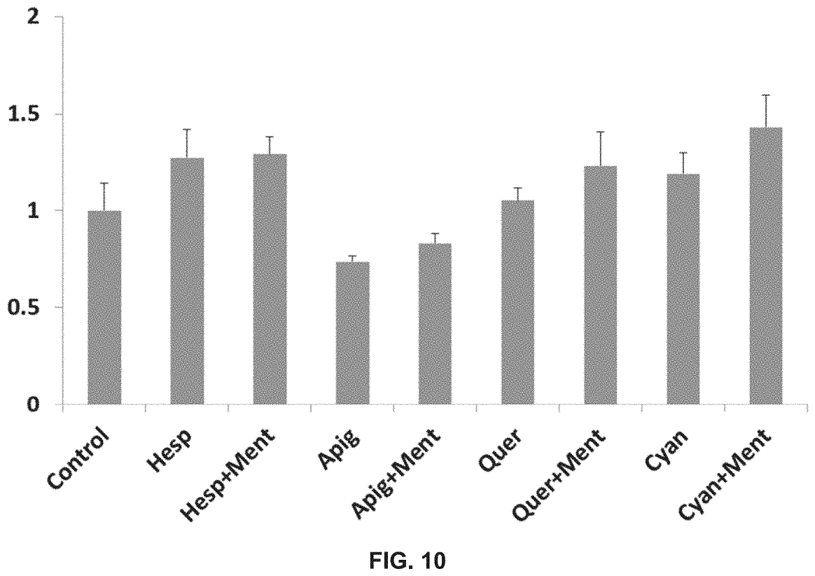

Naringenin was combined with another natural compound, Eucalyptol, shown to activate TRPM8 at 10-4M (Vriens J, et al. Curr Neuropharmacol 2008 6:79-96). Naringenin 5 uMin combination with eucalyptol at 100 μM and 200 μM did not increase UCP1 mRNA expression above the level observed with naringenin alone (FIG. 8). Icilin, a well characterized potent TRPM8 activator, was also evaluated (Journigan V B, et al. Life Sci 2013 92:425-37). Icilin has an EC50 of approximately 0.5 μM, at least 100-fold more potency than menthol. Naringenin 5 μM combined with 0.5 μM icilin did not display synergy for UCP1 mRNA induction (FIG. 9). Therefore, the synergy between naringenin and menthol is selective for menthol.

### Example 4. Analysis of Other Classes of Flavonoids to Synergize with Menthol

Flavonoids from multiple classes were tested with menthol for synergistic induction of UCP1 mRNA. Hesperetin (a flavonone like naringenin), apigenin (flavone), quercetin (flavonol), and cyanidin (anthocyanidin) at 5 μM concentration were each combined with 20 μM menthol for a 7 day treatment of human adipocytes under the same conditions used to induce synergy with naringenin. No statistical difference in UCP1 expression was observed in comparison with each flavonoid alone. Therefore, the menthol synergy is selective for naringenin.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A composition comprising a phosphatidylinositol-4,5-bisphosphate 3-kinase (PI3K) agonist; a transient receptor potential cation channel subfamily M member 8 (TRPM8) agonist; and a pharmaceutically, nutraceutically, or cosmetically acceptable carrier.

2. The composition of claim 1, wherein the TRPM8 agonist comprises menthol.

3. The composition of claim 1, wherein the PI3K agonist comprises naringenin or naringin.

4. The composition of claim 1, wherein the naringenin is in an amount of between about 0.1 μM to about 10 μM, and wherein the menthol is in an amount of between about 1 μM to about 30 μM.

5. The composition of claim 1, wherein the ratio of menthol to naringenin is about 4:1.

6. The composition of claim 1, wherein the naringenin is derived from citrus rinds.

7. The composition of claim 1, wherein the menthol is derived from a mint plant or sunflower essential oil.

8. The composition of claim 1, wherein the menthol is synthetic.

9. The composition of claim 1, wherein the carrier is suitable for topical administration.

10. A method of treating diabetes, obesity, or metabolic syndrome in a subject, comprising administering to a subject in need thereof a therapeutically effective amount of a composition comprising a PI3K agonist and a TRPM8 agonist.

11. The method of claim 10, wherein the composition comprises the composition of any one of claims 1 to 6.

12. The method of claim 10, wherein the composition is selectively administered to body locations of the subject where fat loss is preferred.

13. The method of claim 12, wherein the subject is undergoing a weight loss program.

14. The method of claim 13, wherein the weight loss program comprises diet, exercise, surgery, obesity medications, or a combination thereof.

15. A method of treating a skin condition in a subject, comprising topically administering to a subject in need thereof an effective amount of a composition comprising a PI3K agonist and a TRPM8 agonist.

16. The method of claim 15, wherein the composition comprises a phosphatidylinositol-4,5-bisphosphate 3-kinase (PI3K) agonist; a transient receptor potential cation channel subfamily M member 8 (TRPM8) agonist; and a pharmaceutically, nutraceutically, or cosmetically acceptable carrier.

17. The method of claim 15, wherein the composition further comprises retinol or a retinyl ester.

18. The method of claim 17, wherein the retinyl ester is selected from the group consisting of retinyl palmitate, retinyl acetate, retinyl propionate, retinyl linoleate and mixtures thereof.

19. The method of claim 15, wherein skin conditions is selected from the group consisting of dry skin, photodamaged skin, appearance of wrinkles, age spots, aged skin, acne, skin lightening, psoriasis, atopic dermatosis, and sebum secretion.
